(19) United States
(12) Patent Application Publication
(10) Pub. No.: US 2018/0263495 A1
(43) Pub. Date: Sep. 20, 2018

(54) SECURE PULSE OXIMETER, MONITOR AND CLOUD CONNECTION

(71) Applicant: KONINKLIJKE PHILIPS N.V., EINDHOVEN (NL)

(72) Inventors: John CRONIN, BONITA SPRINGS, FL (US); Jonathan GOGUEN, BONITA SPRINGS, FL (US); Christopher HUFFINES, BONITA SPRINGS, FL (US)

(21) Appl. No.: 15/760,332

(22) PCT Filed: Sep. 26, 2016
(86) PCT No.: PCT/EP2016/072848

§ 371 Date: Mar. 15, 2018

Related U.S. Application Data
(60) Provisional application No. 62/233,612, filed on Sep. 28, 2015.

(30) Foreign Application Priority Data
Mar. 22, 2016 (EP) 16161496.1

(51) Int. Cl. A61B 5/00; A61B 5/1455; G16H 40/67; H04L 9/06; H04L 9/08; H04L 9/32; A61B 5/024

(52) U.S. Cl. CPC A61B 5/0022; A61B 5/14551; G16H 40/67; H04L 9/0662; A61B 5/082; H04L 9/3213; A61B 5/024; A61B 2503/045; A61B 2505/01; H04L 9/0894

(57) ABSTRACT

A method for transmitting and receiving pulse oximetry data comprising: transmitting pulse oximeter sensor data to a pulse oximeter monitor; encrypting the transmitted pulse oximeter data by the pulse oximeter monitor; transmitting the encrypted data to a cloud-based medical service; sending back the encrypted data from the cloud-based medical service to the pulse oximeter monitor, where the data is decrypted and displayed; 5 generating a random number by the pulse oximeter monitor via a random number generator that is triggered by a random number on the sensor, creating a unique ID for a device pulse oximeter monitor for a device; and sending the unique ID over the cloud for each event.

### BACKGROUND

Pulse oximetry is an effective and non-invasive method of monitoring of and acquiring oxygen saturation (SpO2) level and perfusion index of a patient. It is very useful in many situations where monitoring a patient's oxygenation level is important. For example, pulse oximetry is useful in emergency care situations, surgery, post-anesthetic care, and monitoring oxygenation of newly-born infants. Typically, however, data acquired from medical or health-related measurements or tests conducted on a patient may need to be transferred to another device such as a database either within a health care facility or to an external network server. In that case, it is imperative that the medical data be transmitted to another device in a secure way to prevent unauthorized access to the patient's medical data.

### SUMMARY OF THE CLAIMED INVENTION

Pulse oximeter sensor data is communicated to the pulse oximeter monitor. The monitor encrypts the data before sending the data to a cloud medical server. Encrypted data is sent from the cloud medical server back to the pulse oximeter monitor, where the data is decrypted and displayed. The Pulse Oximeter Monitor device has a random number generator that is triggered by a random number on the sensor, so a unique key is created for that pulse oximeter monitor for that device, which is a unique ID, the cloud there receives a unique key for each event.

To ensure patient data remains private and confidential when using cloud services. Pulse oximeter sensor data is communicated to the pulse oximeter monitor. The monitor encrypts the data before sending the data to a cloud medical server. The cloud medical server performs a task, and then encrypted data is sent back from the cloud medical server back to the pulse oximeter monitor, where the data is decrypted and displayed for the patient or medical personnel use.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 shows the system according to a preferred embodiment of the invention.

FIG. 2 is a flowchart illustrating a method according an exemplary embodiment of the present invention.

FIG. 3A shows a flowchart of the sending security software according to a preferred embodiment of the invention.

FIG. 3B shows a flowchart of the receiving security software according to a preferred embodiment of the invention.

FIG. 4A shows a flowchart of the network decrypting software according to a preferred embodiment of the invention.

FIG. 4B shows a flowchart of the network encrypting software according to a preferred embodiment of the invention.

### DETAILED DESCRIPTION

Some embodiments of the invention relate to a method for transmitting and receiving pulse oximetry data comprising: transmitting pulse oximeter sensor data to a pulse oximeter monitor; encrypting the transmitted pulse oximeter data by the pulse oximeter monitor; transmitting the encrypted data to a cloud-based medical service; sending back the encrypted data from the cloud-based medical service to the pulse oximeter monitor, where the data is decrypted and displayed; generating a random number by the pulse oximeter monitor via a random number generator that is triggered by a random number on the sensor, creating a unique ID for a device pulse oximeter monitor for a device; and sending the unique ID over the cloud for each event.

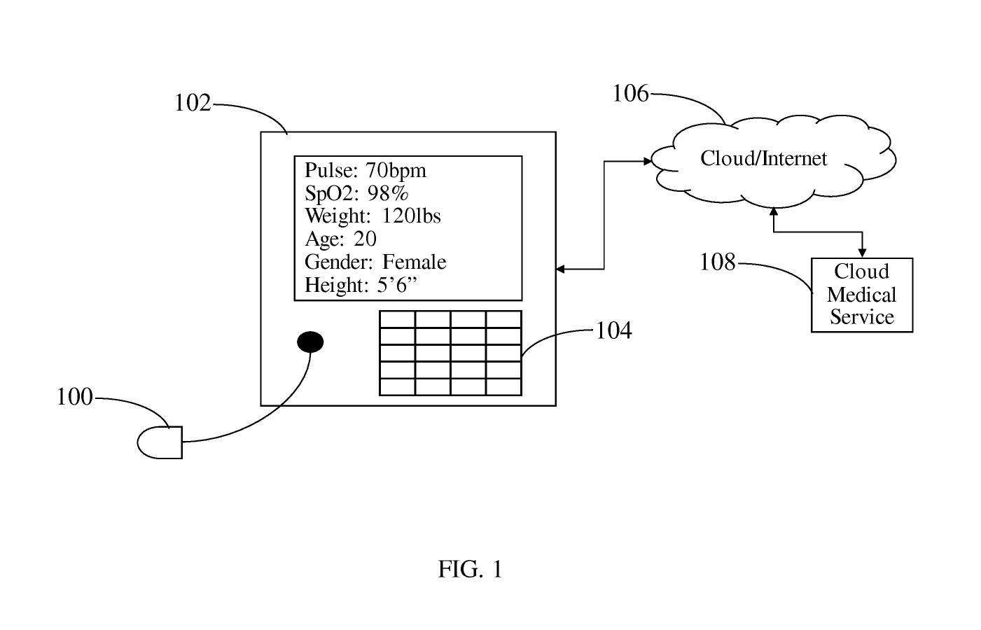

FIG. 1 shows the system according to a preferred embodiment of the invention. A pulse oximeter sensor 100 is connected to a pulse oximeter monitor 102 that has a keyboard 104. The pulse oximeter monitor 102 is connected to a cloud-based medical service 108 via the cloud or internet 106.

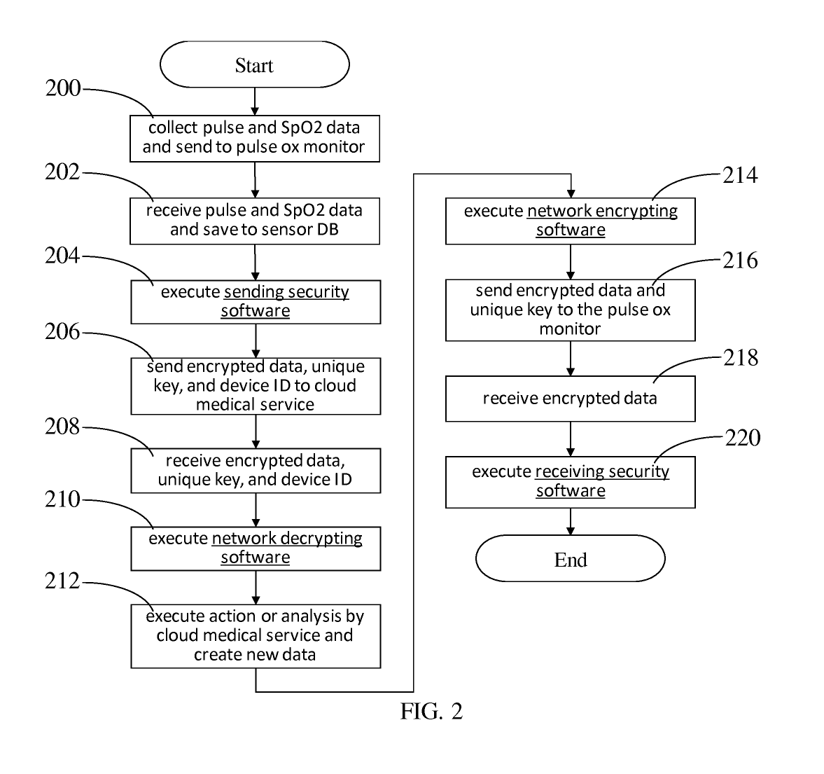

FIG. 2 is a flowchart illustrating the method according to an exemplary embodiment of the present invention. The pulse oximeter sensor collects pulse and SpO2 data and sends the data to a pulse oximetry monitor (step 200). The pulse oximetry monitor then receives pulse and SpO2 data and saves the data to a sensor database (step 202). A sending security software is then executed (step 204) and the resulting encrypted data, a first unique key, and Device ID are transmitted to a cloud medical server (step 206). The cloud-based medical service then receives the encrypted data, the first unique key, and Device ID (step 208). The cloud-based medical service then executes a network decrypting software (step 210). An action or analysis software is executed by the cloud-based medical service thus creating new data (212). The cloud medical server then executes a network encrypting software (step 214) and generates an encrypted new data and a second unique key. The cloud-based medical service then sends back to the pulse oximeter monitor the encrypted new data and the second unique key (step 216). The pulse oximeter monitor receives encrypted data (step 218) and then executes receiving security software (step 220) to decrypt the encrypted data so the received encrypted new data can be read on the pulse oxymetry monitor.

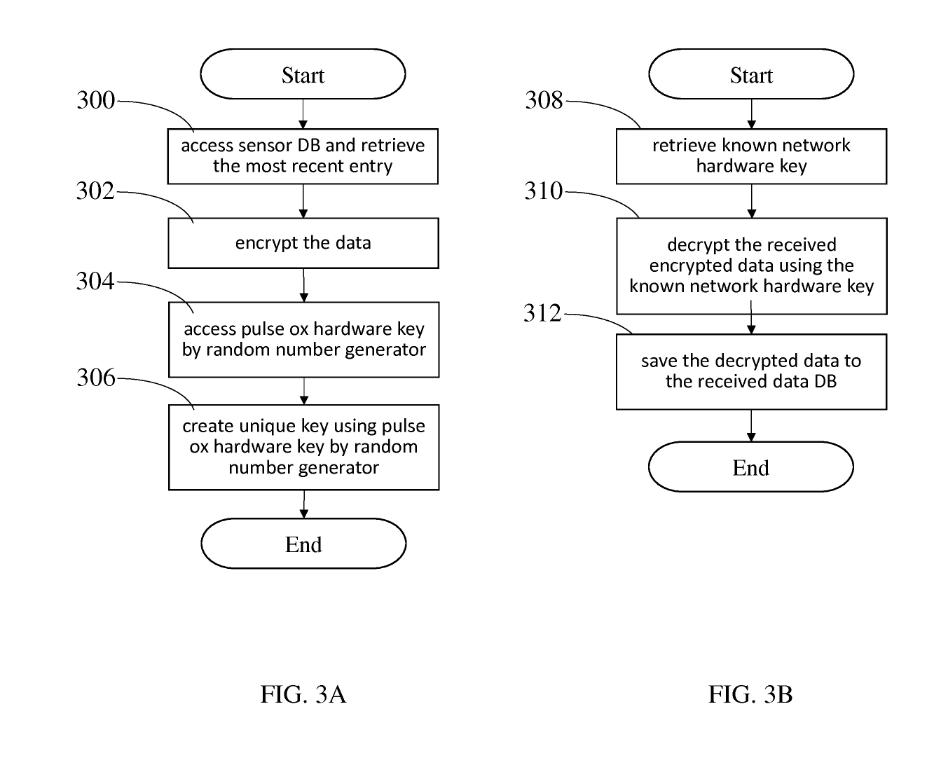

FIG. 3A shows a flowchart of the sending security software according to a preferred embodiment of the invention. In accordance with this embodiment, the sensor database is accessed and the most recent entry of the pulse data and the SpO₂ data is retrieved (step 300). The retrieved data is then encrypted (step 302) and a random number generator then accesses a pulse oximeter hardware key (step 304). Then, the random number generator creates a first unique key using the pulse oximeter hardware key (step 306). FIG. 3B shows a flowchart of the receiving security software according to a preferred embodiment of the invention. Here, the first step involves retrieving a known network hardware key (step 308). The received encrypted data is then decrypted using the known network hardware key (step 310). Then, the decrypted data is saved in the received data database (step 312).

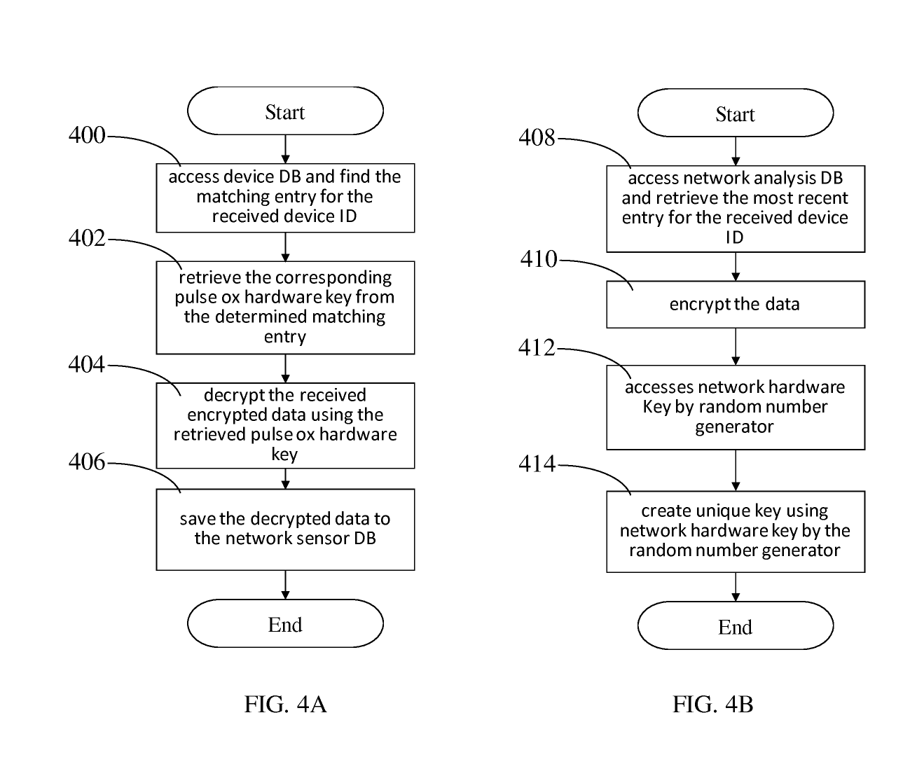

FIG. 4A shows a flowchart of the network decrypting software according to a preferred embodiment of the invention. In the first step, the device database is accessed and the matching entry of the pulse data and the SpO₂ data for the received device ID is located (step 400). The corresponding pulse oximetry hardware key is then retrieved from the determined matching entry (step 402). Next, the received encrypted data is decrypted using the retrieved pulse oximetry hardware key step 404). Then, the decrypted data is saved in the network sensor database (step 406).

FIG. 4B shows a flowchart of the network encrypting software according to a preferred embodiment of the invention. Here, the first step involves accessing a network analysis database and retrieving the most recent entry of the pulse data and the SpO₂ data for the received device ID (step 408). The data is then encrypted (step 410) and the random number generator accesses the network hardware key (step 412). Then, the random number generator creates a second unique key using a network hardware key (step 414).

The confidential medical data can be transmitted and received by any number of types of devices. Examples of these devices are the medical devices that were used to measure data and conduct tests on the patient, as well as devices connected to these medical devices such as computers and storage devices. Thus, in addition to a pulse oximeter, patient medical data may originate or be received by a capnometer, CO-oximeter, arterial blood gas analyzer, glucose meter, and blood pressure monitor. These devices may transmit or receive data using different communication protocols and formats. invention.

Confidential medical data can be transmitted and received by any number of types of devices. Examples of these devices are the medical devices that are used to measure data and conduct tests on the patient, as well as devices connected to these medical devices such as computers and storage devices. Thus, in addition to a pulse oximeter, patient medical data may originate or be received by a capnometer, CO-oximeter, arterial blood gas analyzer, glucose meter, and blood pressure monitor. These devices may transmit or receive data using different communication protocols and formats.

In a preferred embodiment of the invention, any device that exchanges data with the cloud-based medical service is authenticated to restrict access to confidential patient information provided to authenticated medical devices and authorized entities. By requiring authentication, medical data can be protected from being unintentionally broadcast and viewed by unintended recipients.

The device of the present invention can be authenticated using various authentication means. One way to perform authentication is to require an identification or authorization code from the device. The identification code can be any key, number, code, or identifier that permits the device to transmit or receive patient information. In one exemplary embodiment of the present invention, a device transmitting or receiving patient information initially receives and stores an authorization code from an authenticating entity. In response to a request for authentication, the device transmits the authorization code to the authenticating device or entity. If the transmitted authorization code does not match a code stored by the receiver of the patient information (such as a medical data server, remote patient monitoring system, or other device, network, or entity), the patient information will not be accepted from the device that is attempting to transmit the confidential medical data.

The confidential patient information is preferably encrypted before transmitting to the cloud-based medical service. If the data were collected from different devices, the data from each device may be separately encrypted prior to being combined, or the data from the different devices may first be combined and then encrypted as a group. In some cases, it might be desirable to encrypt only certain portions of the data collected from a device or group of devices, e.g., one may want to encrypt only sensitive information relating to the patient to facilitate data transmission or reduce computing requirements.

Alternatively, the encryption process may make use of a private key of a medical device or use of a public key of the intended recipient such as the cloud-based medical service, or both private and public keys. The private and public keys may be transmitted to the cloud-based medical service using wired or wireless communication systems. In one embodiment, the transmitting or receiving entity may request that the public key of the cloud-based medical service be forwarded to it to enable, for example, the decryption of the received encrypted patient medical data. Alternatively, a server that serves as an intermediary between, for example, a hospital server and a cloud-based medical service can retransmit encrypted a patient's medical data from the hospital server to the cloud-based medical service, and vice versa.

If medical data, such as pulse oximetry data, was taken by a patient outside a hospital, for example, the patient will be required to transmit only encrypted information to the cloud-based medical service using any of the previously described encryption and authentication means. In addition to any required key, code, or number, the patient may also be required to transmit additional identifiers appropriate to the patient such as the patient's name, phone number, or social security number.

In an embodiment of the present invention, a server or network such as a cloud-based medical service generates a request to authenticate access. The medical data system then sends a request to authenticate access to a user component of the present invention associated with the patient, user, or health care provider. After receiving the request to authenticate access, the user generates an authentication token.

The authentication tokens may contain text strings or numbers corresponding to patient-related personal information such as phone number, social security number, account number or other patient identifier that can be compared with previously stored patient information in a medical data server. Alternatively, authentication tokens may consist of encoded passwords or biometric data of the patient rendered into a digital representation. Once generated, for security purposes the authorization token may be secured by encrypting the token, digesting and encrypting the digest of the token, or cryptographically hashing the token before transmission to the requesting entity such as the medical data system or server. In one embodiment of the present invention, when authentication tokens are generated, the token sender may create a certification of validity via, for example, encrypting the token with a private key associated with the token originator; encrypting the token with a public key associated with the token requester or destination; generating a digest of the token through a method such as a hashing algorithm and optionally encrypting the hashed digest with the token originator's private key, or providing an authentication code as at least part of the token (such as a cryptographically hashed password) that may be compared to previously stored values. Then, the secured authentication token is sent and when the recipient receives the token along with any encrypted or cleartext certification data, the component may determine the access is valid by attempting to: decrypt an encrypted token with the alleged originator's public key; decrypt an encrypted token with the alleged originator's public key; decrypt an encrypted digest with the alleged originator's public key, and comparing the result to a hashed value of the PIN, token, pin, password, code or comparing a cryptographically hashed password for the alleged originator to known pre-stored values, and if a match is found, authorization is granted.

The token recipient or verifier then receives and analyzes the validity of the authentication token. If the authentication token is confirmed to be authentic, such as by comparing the analyzed token data to known, pre-stored values such as the patient or the patient's health care provider's pre-stored hashed password or other patient personal information, then access is successful and the process terminates. After analyzing the authentication token or a message containing or associated with the token, the token recipient and verifier can then decide that access is either permitted or denied, and may communicate this status to the authentication token sender. At that point, the system may repeat the process, allowing the token originator to attempt access again.

In a preferred embodiment of the invention, a request for pulse oximeter measurement is received. The system then performs diagnostic tests to see if the pulse oximeter is inactive or in an idle state. Otherwise it means that the device had been previously initialized. If it is in an idle state, the pulse oximeter is activated to perform primary signal and reference or baseline measurements. The device may then perform continuous measurements and then graph data, or the device may take a discrete number of measurement samples and compute the sample average and then transmit a single measurement.

Other devices may also be used in conjunction with the pulse oximeter. For example, CO-oximeter is a device for detecting hypoxia and works similar to a pulse oximeter. CO-oximeter measures absorption at several wavelengths to distinguish oxy-hemoglobin from carboxyhemoglobin and determine the oxy-hemoglobin saturation even when the patient has carbon monoxide poisoning.

Another device that may be used in conjunction with the pulse oximeter of the present invention is a capnometer, which is an instrument for monitoring breathing rate and adequacy of ventilation. It attaches to the endotracheal tube and measure the carbon dioxide content in the inspired and expired air. It uses an infrared light to measure the amount of light absorbed by carbon dioxide molecules during breathing. It detects changes in carbon dioxide concentrations in patients who are hemodynamically stable, but not critically ill.

Still another device that may be used in conjunction with the pulse oximeter of the present invention is an arterial blood gas (ABG) analysis. This is a blood test using samples extracted from an artery. The test determines the pH of the blood, the partial pressure of carbon dioxide and oxygen, and the bicarbonate level. Many blood gas analyzers will also report concentrations of lactate, hemoglobin, several electrolytes, oxy-hemoglobin, carboxyhemoglobin and methemoglobin. The arterial blood gas analysis determines gas exchange levels in the blood related to lung function.

The present invention is not intended to be restricted to the several exemplary embodiments of the invention described above. Other variations that may be envisioned by those skilled in the art are intended to fall within the disclosure.

1. A method for transmitting and receiving pulse oximetry data of a patient, the method comprising:
collecting a pulse data and a SpO2 data of the patient using a pulse oximeter;
sending the pulse data and the SpO2 data to a pulse oximetry monitor;
saving the pulse data and the SpO2 data to a sensor database;
generating encrypted data based on the pulse data and the SpO2 data, a first unique key, and a device ID;
transmitting the encrypted data, the first unique key, and the device ID to a cloud medical server in communication with the pulse oximetry monitor;
decrypting the encrypted data;
analyzing the decrypted data to generate new data;
encrypting the new data and a second unique key;
sending the encrypted new data and the second unique key to the pulse oximeter monitor; and
decrypting the encrypted new data, wherein the decrypted new data is displayed on the pulse oximetry monitor.

2. The method of claim 1, wherein generating the encrypted data comprises:
accessing the sensor database and retrieving the pulse data and the SpO2 data that are most recently saved for the device ID;
encrypting the retrieved pulse data and the retrieved SpO2 data;
accessing a pulse oximeter hardware key using a random number generator; and
creating the first unique key using the pulse oximeter hardware key.

3. The method of claim 1, wherein decrypting the encrypted new data comprises:
retrieving a known network hardware key;
decrypting the encrypted new data using the known network hardware key; and
saving the decrypted new data in a received data database.

4. The method of claim 1, wherein decrypting the encrypted data comprises:
accessing a device database and locating the pulse data and the SpO2 data that are most recently saved for the device ID;
retrieving a pulse oximetry hardware key that corresponds to the device ID;
decrypting the encrypted data; and
saving the decrypted data in the sensor database.

5. The method of claim 1, wherein generating the encrypted new data comprises:
accessing a network analysis database and retrieving the pulse data and the SpO2 data that are most recently saved for the device ID;
encrypting the retrieved pulse data and the retrieved SpO2 data;
accessing a pulse oximeter hardware key using a random number generator; and
creating the second unique key using the pulse oximeter hardware key.

6. The method of claim 1, further comprising transmitting and receiving confidential medical data via the cloud medical server using one or more additional devices selected from a capnometer, a CO-oximeter, an arterial blood gas analyzer, a glucose meter, a blood pressure monitor, or a combination thereof.

7. The method of claim 6, wherein the additional devices are authenticated to restrict access to confidential patient information provided to an authenticated medical device or an authorized entity.

8. The method of claim 7, wherein the additional devices are authenticated using an authorization code from the additional devices.

9. The method of claim 8, wherein data from each of the pulse oximeter and the additional devices are separately encrypted prior to being combined.

10. The method of claim 8, wherein data from each of the pulse oximeter and the additional devices are first combined and subsequently encrypted as a group.

11. The method of claim 1, further comprising transmitting an additional identifier appropriate to the patient to the cloud medical server.

12. The method of claim 6, wherein the cloud medical server sends a request to authenticate access to the pulse oximeter and the additional devices.

13. The method of claim 12, wherein the pulse oximeter or one of the additional devices generates an authentication token.

14. (canceled)

15. A system for transmitting and receiving pulse oximetry data of a patient, the system comprising:
a pulse oximeter that collects a pulse data and a SpO2 data of the patient;
a pulse oximetry monitor that receives the pulse data and the SpO2 data from the pulse oximeter;
a sensor database having saved thereon the pulse data and the SpO2 data; and
a pulse oximetry processor that executes instructions stored in memory, wherein execution of the instructions by the processor:
generates an encrypted data using the pulse data and the SpO2 data, a first unique key, and a device ID;
transmits the encrypted data, the first unique key, and the device ID to a cloud medical server connected to the pulse oximetry monitor;
decrypts the encrypted data;
analyzes the decrypted data to generate new data;
encrypts the new data and a second unique key;
sends the encrypted new data and the second unique key to the pulse oximeter monitor; and
decrypts the encrypted new data, wherein the decrypted new data is displayed on the pulse oximetry monitor.

16. The system of claim 15, wherein the pulse oximetry processor is programmed to execute instructions to generate the encrypted data using the pulse data and the SpO2 data, a first unique key, and a device ID by operations including:
generating the encrypted data comprises:
accessing the sensor database and retrieving the pulse data and the SpO2 data that are most recently saved for the device ID;
encrypting the retrieved pulse data and the retrieved SpO2 data;
accessing a pulse oximeter hardware key using a random number generator; and
creating the first unique key using the pulse oximeter hardware key.
